(19) United States
(12) Patent Application Publication
(10) Pub. No.: US 2005/0065137 A1
(43) Pub. Date: Mar. 24, 2005

(54) Triamcinolone acetonide and anecortave acetate formulations for injection

(75) Inventors: Rajni Jani, Fort Worth, TX (US); Ernesto J. Castillo, Arlington, TX (US); Wesley Wehsin Han, Arlington, TX (US)

Correspondence Address: Patrick M. Ryan;Alcon Research, Ltd., R&D Counsel Q-148, 6201 So. Freeway, Fort Worth, TX, 76134-2099, US

(73) Assignee: Alcon, Inc.

(21) Appl. No.: 10/933,006

(22) Filed: Sep. 2, 2004

Related U.S. Application Data
(60) Provisional application No. 60/505,386, filed on Sep. 23, 2003.

(51) Int. Cl. A61K 31/56

(52) U.S. Cl. 514/171

(57) ABSTRACT

Injectable compositions of triamcinolone acetonide or anecortave acetate are disclosed. The compositions are particularly suitable for injection into the posterior segment of the eye to treat ophthalmic diseases.

This application claims priority to U.S. Provisional Application, U.S. Ser. No. 60/505,386, filed Sep. 23, 2003.

### BACKGROUND OF THE INVENTION

1. Field of the Invention

The present invention relates to injectable formulations used for treating diseases or conditions of the eye. More particularly, the present invention relates to formulations of the steroid triamcinolone or the cortisene anecortave acetate that are designed for injection into the eye.

2. Description of the Related Art

Injectable compositions containing triamcinolone acetonide have been available for many years. Commercial products include Kenalog®-10 Injection (triamcinolone acetonide injectable suspension, USP) and Kenalog®-40 Injection (triamcinolone acetonide injectable suspension, USP), which are marketed by Bristol-Myers Squibb Co. These products contain 10 mg/ml or 40 mg/ml of trimacinolone acetonide, respectively. According to its package insert, Kenalog-40 Injection is approved for certain intramuscular and intra-articular uses. Where oral therapy is not feasible or is temporarily undesirable in the judgment of the physician, Kenalog-40 Injection is indicated for intramuscular use in certain cases for endocrine disorders, rheumatic disorders, collagen diseases, dermatologic diseases, allergic states, ophthalmic diseases, gastrointestinal diseases, respiratory diseases, hematologic disorders, neoplastic diseases, and edematous state. The specific approved ophthalmic indication is “[s]evere chronic allergic and inflammatory processes involving the eye, such as: herpes zoster ophthalmicus; iritis; iridocyclitis; chorioretinitis; diffuse posterior uveitis and choroiditis; optic neuritis; sympathetic ophthalmia; and anterior segment inflammation. Kenalog-40 Injection is indicated for intra-articular or intrabursal administration, and for injection into tendon sheaths, as adjunctive therapy for short-term administration (to tide the patient over an acute episode or exacerbation) in: synovitis of osteoarthritis; rheumatoid arthritis; acute and subacute bursitis; acute gouty arthritis; epicondylitis; acute nonspecific tenosynovitis; and posttraumatic osteoarthritis.

Recently, the use of Kenalog®-40 Injection to treat diabetic macular edema, has been growing more common. In this use, the product is injected into the vitreous of patients suffering from diabetic macular edema. In some cases, the product is processed by the physician in an attempt to remove the preservative that is present in the Kenalog-40 Injection formulation supplied by Bristol-Myers Squib Co. because the preservative may be irritating in to the vitreous and tissues in the posterior segment of the eye. Additionally, the commercially available product must be used immediately after it is shaken to avoid settling; the package insert reads as follows: “After withdrawal [from the shaken product vial], inject without delay to prevent settling in the syringe.”

Anecortave acetate is a compound known to be useful for treating ocular angiogenesis-related disorders. U.S. Pat. No. 6,011,023 discloses certain compounds, including anecortave acetate, useful for treating and preventing ocular neovascularization. Various formulations are described in the '023 patent, including formulations for sterile intraocular injection.

What is needed is an improved triamcinolone acetonide or anecortave acetate suspension composition that is suitable for injection into the eye, does not settle rapidly, and can be easily injected through a small needle (e.g., 27-gauge or 30-gauge) that offers the potential for a self-sealing puncture wound.

### SUMMARY OF THE INVENTION

The present invention provides improved triamcinolone acetonide and anecortave acetate suspension compositions that are particularly suited for injection into the eye. The improved suspension compositions have excellent settling characteristics, are easily resuspended with gentle-shaking, are preservative-free and surfactant-free, and are capable of being smoothly and easily injected through 30-gauge needles.

Among other factors, the present invention is based on the finding that a suspension composition of triamcinolone acetonide or anecortave acetate that has improved settling characteristics relative to the currently available Kenalog-40 Injection triamcinolone acetonide composition can be obtained without the need to include any surfactant ingredient. The present invention is also based on the finding that such a trimacinolone acetonide or anecortave acetate suspension composition that lacks a surfactant ingredient can also be more easily injected through a 30-ga. cannula than the currently available Kenalog-40 Injection triamcinolone acetonide composition.

### DETAILED DESCRIPTION OF THE INVENTION

Unless indicated otherwise, all ingredient amounts are presented on a % (w/v) basis.

The suspension compositions of the present invention consist essentially of trimacinolone acetonide or anecortave acetate, polyvinylpyrrolidone, a tonicity-adjusting agent, a buffering agent and water for injection.

Triamcinolone acetonide is a steroid that can be made by known methods and is commercially available even in micronized forms. It is important that the triamcinolone acetonide be sized so that mean volume diameter is 4 μm or less, preferably 3 μm or less, with a standard deviation of around 2 μm or less. Sizing techniques, such as ball-milling, are known and can be used to attain these particle size and distribution requirements. The suspension compositions of the present invention contain from 0.1-25% of trimacinolone acetonide, and, if designed for injection into the posterior segment of the eye, are preferably formulated so that they contain 4%, 8%, 16%, or 25% of trimacinolone acetonide. Most preferred are suspension compositions containing 4% or 8% of trimacinolone acetonide.

Anecortave acetate is a known angiostatic cortisene compound. As in the case of triamcinolone acetonide, it is important that the anecortave acetate be sized so that mean volume diameter is 4 μm or less, preferably 3 μm or less, with a standard deviation of around 2 μm or less. Sizing techniques, such as ball-milling, are known and can be used to attain these particle size and distribution requirements. The suspension compositions of the present invention generally contain from 1-16% of anecortave acetate. If the suspension is designed to be injected into the sub-Tenon's region, the concentration of anecortave acetate is preferably from 3-6%, and most preferably 3%. If the suspension is designed to be injected into the vitreous, the concentration of anecortave acetate is preferably such that the injection delivers from 4-50 mg of anecortave acetate.

In addition to triamcinolone acetonide or anecortave acetate, the suspension compositions of the present invention contain polyvinylpyrrolidone in an amount sufficient to enhance the physical stability of the suspension composition and disperse and wet the drug during any drug sizing process. Polyvinylpyrrolidone is commercially available from a variety of sources in different grades and in a number of molecular weights. For example, polyvinylpyrrolidone is available in at least four grades from International Specialty Products (Wayne, N.J.): Plasdone® C-15 (weight avg. MW=8K), C-30 (endotoxin-free, weight avg. MW=58,000, K-29/32 (weight avg. MW=58K) and K-90 (weight avg. MW=1300K). The polyvinylpyrrolidone ingredient included in the compositions of the present invention has a weight average molecular weight of about 5000-1,600,000. Most preferred is polyvinylpyrrolidone having a weight average molecular weight of about 55,000-60,000. The amount of polyvinylpyrrolidone that should be used in the suspension compositions of the present invention varies with the concentration of trimacinolone acetonide or anecortave acetate, but in general will be from 0.5-8%. For compositions containing 4% trimacinolone acetonide, a suitable amount of polyvinylpyrrolidone is 0.5-1.5%, preferably 1.0%. For compositions containing 8% trimacinolone acetonide, a suitable amount of polyvinylpyrrolidone is 1.5-3%, preferably 2%. For compositions containing 16% or 25% trimacinolone acetonide, a suitable amount of polyvinylpyrrolidone is 3-8%, preferably 4-6%. For compositions containing 1-3% of anecortave acetate, a suitable concentration of polyvinylpyrrolidone is 0.5-1.5%, preferably 1.0%.

The compositions of the present invention have a viscosity of 50 cps. or less, preferably 15 cps. or less, and most preferably 10 cps. or less. They settle very slowly and resuspend readily. This relatively low viscosity ensures that the product is easily processed during manufacturing, transfer and filling operations, and is easily extruded through 27-gauge or 30-gauge needles.

In addition to the triamcinolone acetonide or anecortave acetate and polyvinylpyrrolidone ingredients, the compositions of the present invention contain a tonicity-adjusting agent, such as sodium chloride or mannitol. Preferably, the tonicity-adjusting agent is sodium chloride. The tonicity-adjusting agent is present in an amount sufficient to cause the final composition to have an ophthalmically acceptable osmolality (generally about 150-450 mOsm). Preferably, the final composition has an osmolality of 250-350 mOsm, and most preferably, the suspension composition of the present invention has an osmolality of 270-320 mOsm.

If necessary, the suspension compositions of the present invention also contain a pH-adjusting agent to adjust the pH of the compositions to pH 6-8. The suspension compositions contain a buffering agent to maintain the pH of the compositions within the range of pH 6-8, preferably pH 7.0-7.6. Suitable buffering agents include phosphate buffering agents such as monobasic sodium phosphate (dihydrate) and dibasic sodium phosphate (dodecahydrate).

The suspension compositions of the present invention are preferably packaged in unit dose containers, such as glass or plastic vials. The suspension compositions can also be packaged in pre-filled syringes or cartridges. The suspension compositions are preferably packaged in glass vials.

As used herein, injection “into the posterior segment of the eye” includes, but is not limited to, injection into the vitreous body, injection into or beneath the sclera, and injection external to the vitreous and beneath the Tenon's capsule.

In one embodiment, the present invention relates to a method of treating macular edema including but not limited to diabetic macular edema, or retinal vein occlusion, including central and branch retinal vein occlusions, comprising injecting into the posterior segment of the eye a suspension composition that is preservative-free and surfactant-free and that consists essentially of trimacinolone acetonide, polyvinylpyrrolidone, an ionic tonicity-adjusting agent, a buffering agent and water for injection.

In another embodiment, the present invention relates to a method of treating post-surgical inflammation comprising injecting into the anterior segment of the eye a suspension composition that is preservative-free and surfactant-free and that consists essentially of trimacinolone acetonide, polyvinylpyrrolidone, an ionic tonicity-adjusting agent, a buffering agent and water for injection.

In another embodiment, the present invention relates to a method of treating an ophthalmic disease or condition in the posterior segment of the eye, including but not limited to macular degeneration, comprising injecting into the posterior segment of the eye a suspension composition that is preservative-free and surfactant-free and that consists essentially of anecortave acetate, polyvinylpyrrolidone, an ionic tonicity-adjusting agent, a buffering agent and water for injection.

Certain embodiments of the invention are illustrated in the following examples.

### EXAMPLES 1-3

### Injectable Triamcinolone Acetonide Formulations

TABLE 1

| | %(w/v) | | |
|---|---|---|---|
| Ingredients | Ex. 1 | Ex. 2 | Ex. 3 |
| Triamcinolone acetonide | 4.0 | 8.0 | 16.0 |
| | (40 mg/mL) | (80 mg/mL) | (160 mg/mL) |
| Povidone | 1.0 | 2.0 | 4.0 |
| Sodium Chloride | 0.76 | 0.76 | 0.76 |
| Monobasic sodium | 0.05 | 0.05 | 0.05 |
| phosphate, dihydrate | | | |
| Dibasic sodium | 0.5 | 0.5 | 0.5 |
| phosphate, dodecahydrate | | | |
| NaOH/HCl | QS to pH 7.4 | QS to pH 7.4 | QS to pH 7.4 |
| Water for injection | QS to 100.0 | QS to 100.0 | QS to 100.0 |

A representative compounding procedure for the compositions of this Example is provided below.

Compounding Procedure

Prior to compounding, all glassware and equipment used in formulating are heat sterilized. Dissolve polyvinylpyrrolidone in water for injection, then add the required amount of trimacinolone acetonide and ball-milling beads (e.g., zirconium beads). Steam-sterilize the polymer solution/drug/bead mixture and mill using a ball-mill at 60 RPM for at least 18 hrs. In a separate container dissolve sodium chloride, monobasic sodium phosphate and dibasic sodium phosphate in water for injection. Sterile-filter the salt solution through a 0.2 micron filter membrane. Aseptically, separate drugs and beads in a Buchner filter, rinse zirconium beads first with the salt solution and then with water for injection. Aseptically check/adjust pH and adjust to final weight. Fill the suspension in the proper packaging under sterile conditions.

### COMPARATIVE EXAMPLE 1

### Kenalog®-40 Triamcinolone Acetonide (Bristol-Myers Squibb/Apothecon)

TABLE 2
Kenalog ®-40 Injection Composition as Disclosed on Product Label

| Ingredients | %(w/v) | Function |
|---|---|---|
| Triamcinolone acetonide | 4.0 (40 mg/mL) | Active |
| Carboxymethylcellulose | 0.75% | Suspending agent |
| sodium | | |
| Polysorbate 80 | 0.04% | Surfactant |
| Sodium chloride | QS to isotonicity | Tonicity |
| Benzyl alcohol | 0.99% | Preservative |
| NaOH/HCl | QS to pH 5.0-7.5 | pH adjustment |
| Water for injection | Required volume | |

### EXAMPLE 4

### Settling Study

The compositions of Examples 1-3 and Comparative Example 1 were evaluated to determine their settling characteristics. After preparing the compositions, each was transferred to a graduated cylinder and stored at room temperature. Visual observations were made at the time points indicated in Table 3 below and the sedimentation volume ratio (%) was recorded. Sedimentation volume ratio (%) was calculated as follows: (sedimentation volume/total volume)×100.

TABLE 3
Sedimentation Volume Ratio (%)

| | Evaluation Time (min) | | | | | |
|---|---|---|---|---|---|---|
| | 0 | 5 | 10 | 20 | 40 | 60 |
| Comparative | 100 | 100 | 99 | 97 | 12 | 11 |
| Ex. 1 | | | | | | |
| (Kenalog-40) | | | | | | |
| Ex. 1 | 100 | 100 | 100 | 100 | 100 | 100 |
| (40 mg/mL) | | | | | | |
| Ex. 2 | 100 | 100 | 100 | 100 | 100 | 100 |
| (80 mg/mL) | | | | | | |
| Ex. 3 | 100 | 100 | 100 | 100 | 97 | 97 |
| (160 mg/mL) | | | | | | |

The results in Table 3 show a dramatic change in the physical stability (settling) of the composition of Comparative Example 1 between 20 and 40 minutes after standing at room temperature. In contrast, the suspension compositions of the present invention (Examples 1-3) showed no such dramatic settling, with the suspension compositions of Examples 1 and 2 remaining 100% homogeneous through the 60-minute testing period.

### EXAMPLE 5

### Evaluation of Extrusion Force

The compositions of Examples 1-3 and Comparative Example 1 were evaluated to determine their ‘syringeability’—the relative ease with which they could be extruded through a needle of a given size. The compositions of Examples 1-3 and Comparative Example 1 were tested using an Instron machine (Model 4501; Load Cell Model 2525-807, capacity 22.48 lbs., used for all samples except Comp. Ex. 1; Load Cell Model 2518-805, capacity 1124 lbs., used for Comp. Ex. 1 samples) to determine the amount of force (pound foot) required to extrude them from syringes using two needle sizes: 27-ga. and 30-ga. The rate of expression was kept constant at either of two (calculated) speeds: fast (Instron head 8.8 mL/min. or 20 in./min) or slow (Instron head 0.85 mL/min. or 1.93 in./min.). BSS® (Balanced Salt Solution) irrigating solution was used as a control. The average results from ten samples of each composition and control solution are shown in Table 4.

TABLE 4

| | Force (lb. ft) | | | |
|---|---|---|---|---|
| | Extrusion Speed | | | |
| | Fast (8.8 mL/min.) | | Slow (0.85 mL/min.) | |
| | Needle Size | | | |
| | 30-ga. | 27-ga. | 30-ga. | 27-ga. |
| Ex. 1 (40 mg/mL) | 1.7 | 1.0 | 0.3 | 0.3 |
| Ex. 2 (80 mg/mL) | 2.1 | 1.2 | 0.3 | 0.3 |
| Ex. 3 (160 mg/mL) | 3.6 | 1.7 | 0.7 | 0.4 |
| Comp. Ex. 1 | 6.2^{a,b} | 1.4^{a} | 14.7^{a,c} | 0.8^{d} |
| (40 mg/mL) | | | | |
| BSS ® solution | 1.5 | 0.7 | 0.3 | 0.4 |
| (control) | | | | |

^{a}Because of higher resistance, the higher load cell (Model 2518-805) and a luer-lok syringe had to be used. The results are comparable because the inside diameter of all syringes used in this experiment was the same.
^{b}Wide variation of results: 2.4 to 17.5 lb. ft.
^{c}Several syringes plugged up.
^{d}One of the samples blew the needle off.

### EXAMPLE 6

### Other Physical Characteristics

Viscosity, average particle size, and resuspendability were determined for the compositions of Examples 1-3 and Comparative Example 1. Viscosity was determined using a Brookfield viscometer (CP-42 at 30 RPM). Resdispersibility was determined by visual inspection of hand-shaken samples. The results are shown in Table 5.

TABLE 5

| | Ex. 1-3 | Comp. Ex. 1 |
|---|---|---|
| Viscosity (cps) | 2 (Ex. 1: 40 mg/mL) | 18 |
| | 7 (Ex. 3: 160 mg/mL) | |
| Re-dispersibility (sec) | ca. 5 | ca. 5 |

### EXAMPLES 7 AND 8

### Injectable Anecortave Acetate Formulations

TABLE 6

%(w/v)

| Ingredients | Ex. 7 | Ex. 8 |
|---|---|---|
| Anecortave Acetate | 1.0 | 3.0 |
| | (10 mg/mL) | (30 mg/mL) |
| Povidone | 1.0 | 1.0 |
| Sodium Chloride | 0.76 | 0.76 |
| Monobasic sodium | 0.05 | 0.05 |
| phosphate, dihydrate | | |
| Dibasic sodium | 0.5 | 0.5 |
| phosphate, dodecahydrate | | |
| NaOH/HCl | QS to pH 7.4 | QS to pH 7.4 |
| Water for injection | QS to 100.0 | QS to 100.0 |

A representative compounding procedure for the compositions of this Example is provided below.

Compounding Procedure

Prior to compounding, all glassware and equipment used in formulating are heat sterilized. Dissolve polyvinylpyrrolidone in water for injection, then add the required amount of anecortave acetate and ball-milling beads (e.g., zirconium beads). Steam-sterilize the polymer solution/drug/bead mixture and mill using a ball-mill at 60 RPM for at least 18 hrs. In a separate container dissolve sodium chloride, monobasic sodium phosphate and dibasic sodium phosphate in water for injection. Sterile-filter the salt solution through a 0.2 micron filter membrane. Aseptically, separate drugs and beads in a Buchner filter, rinse zirconium beads first with the salt solution and then with water for injection. Aseptically check/adjust pH and adjust to final weight. Fill the suspension in the proper packaging under sterile conditions.

### EXAMPLE 9

### Settling Study

The compositions of Examples 7 and 8 were evaluated to determine their settling characteristics. After preparing the compositions, each was transferred to a graduated cylinder and stored at room temperature. Visual observations were made at the time points indicated in Table 7 below and the sedimentation volume ratio (%) was recorded. Sedimentation volume ratio (%) was calculated as follows: (sedimentation volume/total volume)×100.

TABLE 7
Sedimentation Volume Ratio (%)

Evaluation Time (min)

| | 0 | 45 | 75 | 120 | 240 |
|---|---|---|---|---|---|
| Ex. 7 | 100 | 100 | 100 | 100 | 100 |
| Ex. 8 | 100 | 100 | 100 | 100 | 100 |

The results in Table 3 above show a dramatic change in the physical stability (settling) of the composition of Comparative Example 1 between 20 and 40 minutes after standing at room temperature. In contrast, the results in Table 7 for the suspension compositions of the present invention (Examples 7 and 8) showed no such dramatic settling, with the suspension compositions of Examples 7 and 8 remaining 100% homogeneous through the 240-minute testing period.

### EXAMPLE 10

### Evaluation of Extrusion Force

The compositions of Examples 7 and 8 were evaluated to determine their ‘syringeability’—the relative ease with which they could be extruded through a needle of a given size. The compositions were tested using an Instron machine (Model 4501; Load Cell Model 2525-807, capacity 22.48 lbs., used for all samples) to determine the amount of force (pound foot) required to extrude them from syringes using two needle sizes: 27-ga. and 30-ga. The rate of expression was kept constant at either of two (calculated) speeds: fast (Instron head 8.8 mL/min. or 20 in./min) or slow (Instron head 0.85 mL/min. or 1.93 in./min.). The samples were loaded into a tuberculin syringe by withdrawing them through an 18-ga. needle. After filling the syringe to approximately the 1 cc level, the 18-ga. needle was removed and either the 30-ga. or 27-ga. needle was attached. The syringe was then placed in the Instron machine and the extrusion force was measured. Ten determinations were made for each sample at each needle size and at each speed and an average value was determined (except as noted). The data is presented in Table 8 below.

TABLE 8

| | Force (lb. ft) | | | |
|---|---|---|---|---|
| | Extrusion Speed | | | |
| | Fast (8.8 mL/min.) | | Slow (0.85 mL/min.) | |
| | Needle Size | | | |
| | 30-ga. | 27-ga. | 30-ga. | 27-ga. |
| Ex. 7 (10 mg/mL) | 1.5 | 0.8 | 0.3^{a} | 0.3 |
| Ex. 8 (30 mg/mL) | 1.7 | 0.8 | 0.4 | 0.2 |

^{a}Four high outliers were discarded by 4 s.d. rule.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

1. A suspension composition particularly suited for injection into the eye, wherein the suspension composition does not contain a preservative or surfactant, and has a pH from 6-8 and a viscosity of 50 cps. or less and wherein the suspension composition consists essentially of:
a) trimacinolone acetonide or anecortave acetate;
b) polyvinylpyrrolidone in an amount sufficient to enhance the physical stability of the suspension composition;
c) a tonicity-adjusting agent in an amount sufficient to cause the suspension composition to have an osmolality from 150-450 mOsm;
d) a buffering agent;
e) water for injection; and
f) optionally a pH-adjusting agent to adjust the pH to 6-8.

2. The suspension composition of claim 1 wherein the suspension composition contains trimacinolone acetonide.

3. The suspension composition of claim 2 wherein the concentration of triamcinolone acetonide is from 0.1-25% (w/v).

4. The suspension composition of claim 3 wherein the concentration of triamcinolone acetonide is 4% (w/v).

5. The suspension composition of claim 3 wherein the concentration of triamcinolone acetonide is 8% (w/v).

6. The suspension composition of claim 3 wherein the concentration of triamcinolone acetonide is 16% (w/v).

7. The suspension composition of claim 1 wherein the trimacinolone acetonide has a mean volume diameter of 4 μm or less, with a standard deviation of 2 μm or less.

8. The suspension composition of claim 1 wherein the suspension composition contains anecortave acetate.

9. The suspension composition of claim 8 wherein the concentration of anecortave acetate is from 1-16% (w/v).

10. The suspension composition of claim 9 wherein the concentration of anecortave acetate is 3-6% (w/v).

11. The suspension composition of claim 8 wherein the anecortave acetate has a mean volume diameter of 4 μm or less, with a standard deviation of 2 μm or less.

12. The suspension composition of claim 1 wherein the polyvinylpyrrolidone has a weight average molecular weight of 55,000-60,000.

13. The suspension composition of claim 1 wherein the tonicity-adjusting agent is sodium chloride.

14. The suspension composition of claim 1 wherein the amount of polyvinylpyrrolidone is 0.5-8% (w/v).

15. The suspension composition of claim 1 wherein the buffering agent comprises monobasic sodium phosphate, dihydrate and dibasic sodium phosphate, dodecahydrate.

16. A method of treating macular edema or retinal vein occlusion in an eye comprising injecting into the posterior segment of the eye the suspension composition of claim 2.

17. A method of treating post-surgical inflammation in an eye comprising injecting into the anterior segment of the eye the suspension composition of claim 2.

18. A method of treating an ophthalmic disease or condition in the posterior segment of the eye comprising injecting into the posterior segment of the eye the suspension composition of claim 8.

19. A triamcinolone acetonide suspension composition particularly suited for injection into the posterior segment of the eye, wherein the suspension composition does not contain a preservative or surfactant, and has a viscosity of 10 cps. or less and wherein the suspension composition consists essentially of:
a) 2-16% (w/v) trimacinolone acetonide;
b) 0.5-4% (w/v) polyvinylpyrrolidone;
c) an ionic tonicity-adjusting agent in an amount sufficient to cause the suspension composition to have an osmolality from 250-350 mOsm;
d) a buffering agent comprising monobasic sodium phosphate, dihydrate and dibasic sodium phosphate, dodecahydrate;
e) NaOH or HCl in an amount to adjust the pH of the suspension composition to 7.0-7.6; and
f) water for injection.

20. An anecortave acetate suspension composition particularly suited for injection into the posterior segment of the eye, wherein the suspension composition does not contain a preservative or surfactant, and has a viscosity of 10 cps. or less and wherein the suspension composition consists essentially of:
1-3% (w/v) anecortave acetate;
0.5-1.5% (w/v) polyvinylpyrrolidone;
an ionic tonicity-adjusting agent in an amount sufficient to cause the suspension composition to have an osmolality from 250-350 mOsm;
a buffering agent comprising monobasic sodium phosphate, dihydrate and dibasic sodium phosphate, dodecahydrate;
NaOH or HCl in an amount to adjust the pH of the suspension composition to 7.0-7.6; and
water for injection.
